(19) United States
(12) Patent Application Publication
(10) Pub. No.: US 2025/0210154 A1
(43) Pub. Date: Jun. 26, 2025

(54) REPRESENTING CLINICAL TRIAL DATA IN AN INTERACTIVE ANALYSIS PLATFORM

(71) Applicant: Telperian, Inc., Solana Beach, CA (US)

(72) Inventor: Ryan Hafen, Solana Beach, CA (US)

(21) Appl. No.: 18/990,773

(22) Filed: Dec. 20, 2024

Related U.S. Application Data
(60) Provisional application No. 63/614,517, filed on Dec. 22, 2023.
(60) Provisional application No. 63/614,516, filed on Dec. 22, 2023.

(51) Int. Cl. G16H 10/20; G06F 16/22; G16H 70/40

(52) U.S. Cl. CPC G16H 10/20; G06F 16/2246; G16H 70/40

(57) ABSTRACT

Provided is a process, including: obtaining data assets characterizing a plurality of clinical studies, selecting a first subclass from a hierarchy of classes for the first data asset, selecting a second subclass from the hierarchy of classes for the second data asset, transforming the first data asset into the shared data format and data schema using the respective selected subclass storing transformation data including unique subject identifier mapping, the selected subclass, and the first format and first data schema of the first data asset, and saving resulting transformed first data asset in the shared data format and data schema in memory.

### CROSS-REFERENCE TO RELATED APPLICATIONS

This patent application claims the benefit of U.S. Provisional Application 63/614,517, filed on 22 Dec. 2023, titled REPRESENTING CLINICAL TRIAL DATA IN AN INTERACTIVE ANALYSIS PLATFORM, and claims the benefit of U.S. Provisional Application 63/614,516, filed 22 Dec. 2023, titled DATA INTEGRITY FOR CLINICAL STUDIES. The entire content of each aforementioned filing is hereby incorporated herein by reference.

### BACKGROUND

### 1. Field

The present disclosure relates generally to computer systems for managing data related to clinical studies and, more specifically, to processing clinical trial data in an interactive analysis platform.

### 2. Description of the Related Art

Clinical trial data is often structured and stored in a variety of formats by different pharmaceutical companies, influenced by factors such as the trial stage, company-specific practices, and other variables. This data often originates from case report forms (CRFs), which are electronic or paper documents that capture protocol-related information about participants at various points in the study. Due to the lack of uniformity in data structures and protocols, clinical trial information is difficult to reconcile into a single, standardized format for analysis. The Clinical Data Interchange Standards Consortium (CDISC) has developed data standards, including CDASH for data collection and SDTM/ADaM for data analysis and FDA submission. While the SDTM and ADaM formats are widely adopted because of FDA submission requirements, transforming raw data into these formats is a costly and time-consuming process that typically happens at the end of the trial. As a result, clinical trial data is rarely available in a format that facilitates interactive analysis throughout the trial, and even after transformation, significant variability remains in how different companies represent key data concepts, making ongoing analysis cumbersome.

This variability complicates the ability of pharmaceutical companies to monitor the clinical data as the trial progresses. Safety data is often useful, offering insights into possible side effects, adverse reactions, and associated toxicity levels. Efficacy data often provides the evaluation of whether the drug or device performs as anticipated, often juxtaposed against existing treatments or placebos. There is also often an analysis of how the drug operates within the body, encompassing its absorption, distribution, metabolism, and excretion, known as pharmacokinetic and pharmacodynamic data. Moreover, understanding how a treatment might elevate the overall quality of life for patients may come through analysis of quality-of-life metrics and patient-reported outcomes.

### SUMMARY

The following is a non-exhaustive listing of some aspects of the present techniques. These and other aspects are described in the following disclosure.

The following embodiments may be useful to address the challenges associated with inconsistent data formatting and delayed availability for data analysis. Some embodiments support interactive analysis of clinical trial data using a unified general data model or other data model that provides a structure that can receive mappings from various data formats. The data model allows for the flexible representation of clinical trial data, transforming raw data into a universal format that may allow for minimal adjustments, facilitating its use on the platform for real-time analysis. The model incorporates various data domains and hierarchical data classes, each tailored for specific types of visualizations and analyses. This structure provides the necessary flexibility to handle data from any clinical trial, allowing users to explore and analyze their data interactively throughout the course of the trial, with reduced processing time and effort. While the described embodiments may be useful to address the challenges discussed above, it should not be assumed that all embodiments are designed to address these challenges but may be created to address other undescribed challenges or needs.

Some aspects include a process including: obtaining, with a computer system, data assets characterizing a plurality of clinical studies, wherein a first data asset comprises a first format having a first data schema and a second data asset comprises a second format having a second data schema; selecting, with the computer system, a first subclass from a hierarchy of classes for the first data asset, wherein the first subclass specifies how to transform the first format and first data schema into a shared data format and data schema; selecting, with the computer system, a second subclass from the hierarchy of classes for the second data asset, wherein the second subclass specifies how to transform the second format and second data schema into the shared data format and data schema, the shared data format and data schema comprising: a base dataset class comprising a mapping relating a unique subject identifier; an event dataset class inheriting from the base dataset class; and a subject-level dataset class inheriting from the base dataset class, wherein at least some of the subclasses inherit from a shared parent class in the hierarchy, and each of the subclasses inherit from a root class in the hierarchy; transforming, with the computer system, the first data asset into the shared data format and data schema using the respective selected subclass; storing, with the computer system, transformation data comprising unique subject identifier mapping, the selected subclass, and the first format and first data schema of the first data asset; and saving, with the computer system, the resulting transformed first data asset in the shared data format and data schema in memory.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a flowchart of an example process by which one or more computer systems may transform clinical data assets into a shared data format, in accordance with some embodiments;

FIG. 2 is a flowchart of an example process by which one or more computer systems may transform clinical data assets into a shared data format including a feedback loop, in accordance with some embodiments;

FIG. 3 is an example data structure of the shared data format, in accordance with some embodiments; and

FIG. 4 is an example computing environment within which a clinical data transformation process may be implemented, in accordance with some embodiments.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the field of data transformation, computer science, human-computer interaction, and clinical studies. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Pharmaceutical companies and others want to be able to analyze their data in an interactive manner throughout the course of a clinical trial to evaluate data quality, monitor safety and efficacy, and make decisions about the trial as it proceeds.

Available tools for extracting, transforming, and loading data into systems that address these needs are lacking. Clinical trial data is structured and stored in many different forms by different pharmaceutical companies. Much of the data arrives in the form of a case report form (CRF), which can be an electronic or paper document that records the protocol and information about each participant at different points of the study. Raw data is also received from laboratory testing results and other sources in some cases. Electronic Data Capture (EDC) Systems and other tools are used to help collect and manage the data of ongoing trials, but the way the raw data can be structured and stored varies greatly. These raw formats are also rarely conducive to meaningful analysis and can entail a great deal of transformation to be useful for that purpose.

The Clinical Data Interchange Standards Consortium (CDIS®) has created data standards for data collection (CDASH, Clinical Data Acquisition Standards Harmonization) as well as standards for structuring data for analysis datasets (ADaM, Analysis Data Model) and submission to the United States Food and Drug Administration (FDA) (Study Data Tabulation Model, SDTM/ADaM). Because the SDTM and ADaM formats are required for FDA submission, they are widely adopted by industry. Transforming data to conform to the SDTM/ADaM standards is a costly and time-consuming process that typically does not happen until the end of a trial or close to FDA submission. This means that often the data is not available in a format that is conducive to interactive analysis until the end of the trial in many cases. None of which should be read to suggest that the present techniques are limited to transforming data into formats consistent with the standards, as the present techniques are more general and standards are expected to evolve in the future while still presenting similar issues that can be amenable to being addressed by some of the techniques described below.

Further, even after data has been transformed to conform to CDISC standards, there is often a wide degree of variability with how different concepts embodied by the standards are represented in the data from one company to another, which makes it difficult for data to be fed into any software-implemented platform (or other tool) that can perform automated (e.g., fully automated or user-driven interactive semi-automated) analysis. Instead, pharmaceutical companies often find themselves working with contract research organizations (CRO) to produce one-off analyses or visualizations that require understanding the nuances of the transformed data each time.

It should be appreciated that this discussion of various issues with existing techniques should not be read as a disclaimer of systems that do not comprehensively address every single one of these problems, as various inventive techniques are described herein, and some aspects only address a subset of these issues or other issues.

Some embodiments implement a software as a service (SaaS) interactive clinical trial data analysis platform, to be referred to as Telperian Foundation™ (or “Foundation”). Some embodiments provide a self-service interface (e.g., on a client computing device communicating via the Internet with a server system hosting the service) to allow users to explore their clinical trial data in an interactive manner, allowing them to quickly gain insights and answer questions in some embodiments. However, many existing EDC systems are not suitable to provide such services to a large number of clients with varying data formats. Some embodiments represent the clinical trial data from diverse trials and users in a way that is general and flexible yet provides enough structure to allow for meaningful analysis. Some embodiments implement the below-described shared data model or similar models that are expected to mitigate these challenges. Many existing EDC systems are not well suited to transform raw clinical trial data into a unified general data model or similar models providing the benefits of a unified general data model, in part because of the high degree of variability in schemas and formats of that raw input data, challenges with accommodating such wide range of variation in inputs in algorithms to transform those inputs into the unified general data model format, and computational complexity issues that arise when mitigating these challenges with traditional approaches to transforming diverse formats of input data into a unified format.

The unified general data model used in Foundation is expected to be a flexible, yet structured way to represent clinical trial data. Some embodiments represent data from a diverse variety of clinical trials, regardless of the format of the raw data, with the least (or a relatively low number of) required transformations, thereby expediting computation, reducing latency, and reducing consumption of computing resources. The model, in some embodiments, provides (e.g., in an ontology or taxonomy) different data domains and hierarchies of data classes that build upon each other to represent data that is expected to be suited for different types of analysis and data visualization.

Some embodiments are configured to process and represent raw data in a variety of formats, accommodating data from multiple clinical trials, each potentially organized in different formats and schemas. The raw clinical data, in some embodiments, provided to the model may include datasets from multiple trials, each structured according to the specific requirements of those trials. In some cases, the clinical data may be in a single, consistent format and schema across all data assets, while in other cases, data from different trials may be presented in diverse formats and schemas. The model, in some embodiments, is capable of transforming each piece of raw clinical data into a unified general data model, ensuring standardized representation regardless of the initial format. This flexibility is expected to help the model to process and represent clinical data effectively, whether it is presented in a single format or in multiple, varied formats.

Data formats and data schemas are distinct concepts. A data format may define the structure and representation of data at the storage or transmission level, while a data schema may describe the logical organization, constraints, and relationships within the data. A data format specifies how data is encoded, serialized, or transmitted, often in terms of bytes or textual representation or at higher levels (like a date format, street address format, and the like), and may include rules for parsing and interpreting the raw data. Examples of data formats may include JSON (JavaScript Object Notation), XML (extensible Markup Language), or binary formats such as Protocol Buffers or Avro. These formats may dictate the syntax used to store or communicate data but, in some cases, do not impose strict rules on the data's content beyond basic structural guidelines.

A data schema, on the other hand, defines the logical blueprint of the data, including the types, attributes, relationships, and constraints to be adhered to for the data to be considered valid. For example, a schema for a database table may define the column names, data types (e.g., integer, string), permissible value ranges, and relationships to other tables, such as foreign key dependencies. Schemas are often defined using tools or languages such as XML Schema Definition (XSD) for XML, JSON Schema for JSON, or Structured Query Language (SQL) for relational databases.

While a data format focuses on the low-level representation and interchange of data, a schema provides higher-level rules and context, allowing systems to validate, interpret, and manipulate the data meaningfully. Both may work together, as schemas are often applied to validate or describe data conforming to a specific format. For example, a JSON Schema may validate that a JSON document adheres to predefined rules about its structure and content.

Some embodiments may implement these present techniques using an object-oriented programming approach, where classes are instantiated to represent different datasets. Each dataset class in these embodiments includes a defined set of “mappings.” These mappings serve as associations that link specific variables within a dataset—such as a field in a record for an individual trial participant—to higher-level concepts that are relevant for various analytical and visualization operations. For instance, in clinical trial data, a variable such as “age” or “adverse event severity” is mapped to corresponding analytical concepts (e.g., “demographic data,” “safety indicator”) which are structured to inform specific data transformations or visualizations. The mappings themselves may be defined within each dataset class or subclass using structured metadata that specifies the type of data (e.g., categorical, numeric, date), the expected format, and any transformation rules necessary to standardize the variable for unified analysis. This metadata, in some embodiments, allows the system to interpret the variables consistently, ensuring that data assets from different sources adhere to the unified general data model. Moreover, dataset classes can, in some embodiments, inherit mappings from parent classes in a hierarchical structure, enabling the reuse of standard mappings across different data types. This inheritance feature, in some embodiments, builds up a collection of interrelated mappings, facilitating increasingly complex analyses and visualizations as more specialized subclasses (e.g., specific to adverse events or demographics) add mappings that address unique data attributes pertinent to each domain.

In some embodiments, the step of selecting a data class or subclass within the hierarchical structure may be implemented by one or more machine learning algorithms, configured to evaluate data attributes and identify the most appropriate class based on pre-trained models. Machine learning, in some embodiments, is leveraged to automate and improve the accuracy of class selection, allowing the system to recognize and classify patterns within diverse clinical data formats efficiently. Various machine learning models may be suited to this task, each offering strengths for different types of classification and data transformation requirements. Algorithms such as Random Forest, Support Vector Machine (SVM), k-Nearest Neighbors (k-NN), Decision Tree Classifiers, Neural Networks, Hierarchical Clustering, or Transformer Models may provide distinct advantages for identifying and classifying data assets based on their format and schema.

In some embodiments, the Random Forest algorithm may be leveraged for robust classification of clinical data assets by constructing an ensemble of decision trees, each trained on bootstrapped subsets of the dataset. This ensemble approach, in some embodiments, mitigates overfitting and improves generalization, leveraging the diversity of trees to reduce variance across the classification process. During training (e.g., using CART, or classification and regression tree), each decision tree may be built by selecting a random subset of features at each node—such as schema structure indicators, variable types, and specific field identifiers—helping the model to capture unique decision paths without relying on any single feature set. This approach is expected to be particularly effective in managing high-dimensional data with complex dependencies across hierarchical subclasses. When classifying a new data asset, in some embodiments, the Random Forest extracts features indicative of format-specific or schema-specific characteristics and propagates these through each decision tree independently. At each node of a decision tree, the algorithm may be configured to evaluate a feature based on its impurity reduction, possibly using Gini impurity or entropy metrics to determine optimal splits, by seeking to minimize impurity or entropy on either side of the split. The asset, in some embodiments, traverses down the model tree until reaching a terminal node, where a provisional class assignment is made. Once all trees in the forest have classified the asset, the final class or subclass is determined by majority voting, wherein the class receiving the most votes across all trees is selected, yielding a highly resilient classification even in the presence of noisy or overlapping feature spaces. To maintain high classification accuracy and adaptability, the model may incorporate a feedback loop that continually refines performance based on real-time user interactions and system feedback. This loop may include periodic retraining on new data or user-validated classifications, as well as hyperparameter tuning (e.g., adjusting the ensemble size, maximum depth, or minimum samples per split) to optimize model performance under evolving data distributions.

In another exemplary embodiment, a Support Vector Machine (SVM) algorithm may be employed to classify clinical data assets based on high-dimensional feature vectors derived from schema structures, variable types, and content-specific attributes. The SVM is trained on labeled clinical data, where each data asset is represented as a point in feature space, with the SVM finding an optimal hyperplane that maximizes the margin between distinct classes. For data with non-linear separability, a kernel trick—such as the radial basis function (RBF) or polynomial kernel—may be applied, transforming the feature space to a higher dimension where linear separation is achievable. During classification, the SVM evaluates each data asset by projecting it into the transformed feature space and calculating its distance from the hyperplane. Based on its position relative to the decision boundary, the SVM assigns the asset to a class. This margin-based approach minimizes misclassification, especially when dealing with overlapping feature distributions typical in clinical data with varied formats. Moreover, the SVM's regularization parameter is tuned through cross-validation, balancing margin maximization with error tolerance to ensure robust classification even with noisy data. An adaptive feedback loop may also be integrated into the SVM model, wherein user-provided feedback or new data samples prompt iterative retraining of the classifier. By incorporating this feedback, the SVM continuously refines the decision boundary, adjusting to subtle changes in data patterns that may emerge as new schema formats or variable types are introduced.

In another exemplary embodiment, the k-Nearest Neighbors (k-NN) algorithm may be utilized for classifying clinical data assets based on the proximity of each asset to its k nearest neighbors within a multidimensional feature space. Each data asset is represented by key features—such as specific field names, schema size, and structural attributes—that define its position in the space. The algorithm assigns a class label to a new data asset by examining the classes of its k closest neighbors, where k is a hyperparameter optimized through cross-validation to balance between bias and variance. The k-NN algorithm's classification relies on a distance metric, typically Euclidean distance for continuous features or Hamming distance for categorical data, to determine neighbor proximity. Once the k nearest neighbors are identified, the data asset is classified based on a majority vote of the classes represented among these neighbors. To improve performance in high-dimensional spaces, dimensionality reduction techniques such as Principal Component Analysis (PCA) may be applied to the feature space prior to classification. Additionally, a feedback loop may support the system by updating the k-NN model by adjusting k or recalculating distance metrics based on newly available data or user-validated classifications.

At the base of this data model, in some embodiments, is a “base” (or root) dataset class, base here referring to a hierarchy of data set classes. This class, in some embodiments, only has one mapping, “subjid_var”, which relates to a unique subject identifier, or some embodiments may have more mappings. This is a useful concept for clinical trial data, as often every record of raw clinical trial data corresponds to a subject, with each record having a variety of fields of information gathered in the trial. This mapping may be used to identify (e.g., expressly or pseudonymously) the subject in the data and may be used to link records from different datasets together, e.g., when the same participant is described by records in different data sets. In some cases, a single trial may produce one data set or multiple data sets. Datasets further to the root dataset class may inherit from those closer to the root dataset class through which they connect to the root dataset class.

A set of visualization methods is provided by some embodiments for the “base” dataset class. These may include interactive univariate numeric and categorical summaries for any column (or other field) in the data. Examples of univariate numeric summaries may include, for the respective column, the mean, median, mode, standard deviation, variance, range, quartiles and interquartile range, minimum and maximum values, or histograms. Examples of univariate summaries for categorical data may include, for the respective column, frequency distributions, percentage distributions, mode, bar charts or pie charts, contingency tables, proportions or ratios or the like. Some embodiments may automatically generate one or more of these summaries, in some cases automatically selecting among these types of summaries to choose a summary appropriate for the respective field of data.

Another dataset class, in some embodiments, is an “event”, which inherits from the base class and therefore may include a subject identifier mapping or other mappings contained within the parent class. Event datasets may pertain to the domain of records of events that have been recorded for a patient (or other clinical trial participant), such as medical history or concomitant medications. These datasets may have a structure of one row per event per patient with variables containing information about the event including when it started and ended. Event datasets may have a set of additional mappings that indicate pairs of “start” and “end” variables (e.g., event start date and end date, event start day and end day relative to beginning of study, etc.), a variable that indicates the “name” of each event, and a variable that indicates a “classification” of the event. Visualizations associated with these datasets (and generated by some embodiments automatically) may include an incidence plot and a swimmer plot, which make use of the mappings to determine how to transform and display the data.

In some embodiments, an even more specialized dataset that is built on top of (e.g., inherits from) “event” datasets is the “Adverse Events” class. This dataset class may have all the mappings and expected structure of an events dataset, and may have additional mappings specific to the adverse events domain, including mapping to variables that indicate the severity of the event, whether it was treatment emergent, etc. These mappings are expected to assist with additional computations and visualizations to be performed specific to this domain, such as a treatment emergent overall summary plot and a treatment emergent forest plot, which may be generated automatically in some cases. In some embodiments, additional classes may inherit from the adverse events class. The child classes contain mappings from the adverse event class as well as additional mappings. Some embodiments may include classes inheriting from the adverse events class or additional mappings within the adverse class related to; dose limiting, toxicity, treatment emergent adverse events, non-serious adverse events, severity-based subclasses, organ system specific adverse events, long term follow-up, or quality of life impact.

A dataset class that is used in conjunction with all of the other dataset classes, in some embodiments, is the “subject-level” class. This dataset may have a structure of one row per subject and may contain many mappings that are useful and integrated throughout all other datasets and visualizations. This includes, for example, the primary treatment variable, treatment start and end dates, demographic variables, baseline health indicators, primary diagnosis or condition codes, physical attributes, compliance and adherence metrics, follow up status and dates, concurrent medications and therapies, etc. This dataset is expected to be helpful in some cases in that some or all of the variables in this dataset can be added to any of the other datasets by nature of each row representing a single subject. It may be joined to other datasets using the subject identifier mapping that is present in all datasets.

In some embodiments, while the specified dataset classes, such as the base, event, adverse events, and subject-level classes, provide essential structure for clinical trial data, additional dataset classes may also be utilized to support specialized data handling requirements or unique analytical needs. These supplementary dataset classes can inherit from existing classes, such as the subject-level or event class, and add unique mappings and transformation rules that make them suitable for the specific data they manage. This flexibility, in some embodiments, allows the system to adapt to diverse clinical trial designs and ensures that specialized data assets are integrated within the broader data model.

The data model, including the data hierarchy, mappings, and linking of visualizations to the datasets and mappings, may be represented as JavaScript object notation (JSON) or other data serialization format. This is expected to facilitate integration at some or all layers of the platform. In the pre-processing stage, some embodiments of the data model may be used to help identify whether datasets might belong to a certain class and to validate that the data that comes out of the preprocessing is in the expected format. In the application layer, the data model may be used to present the appropriate visualization choices to the user given the dataset they are looking at, and to define an interface that allows users to edit the mappings.

The data model is expected to support various features of the Telperian Foundation platform, examples of which are described below, including annotations. Embodiments may detect when an issue is captured related to any visualization, and in response, capture that visualization (in its current state, which could otherwise evolve), store it as part of the issue, and display it when displaying the issue. This is expected to provide meaningful context as users collaborate to resolve issues. The visualizations, when preserved, may be specified through the dataset (e.g., by specifying a version thereof) and mappings as well as various other user inputs.

In some embodiments, when an issue is identified related to a visualization, the system captures the visualization in its current state, preserving it as part of the issue record. This captured visualization may include associated metadata to assist in issue resolution. Examples of such metadata include the date and time the visualization was generated, user-specific information (e.g., user ID and role), parameters or filters applied to the visualization, relevant dataset identifiers, and any mappings or transformations that were applied to the underlying data. This metadata, in some embodiments, provides contextual information that allows users to trace back to the precise configuration of the visualization at the time the issue was captured, facilitating a more targeted and efficient resolution process.

In some embodiments, when an annotation is created, the system captures and stores the parameters associated with the visualization, ensuring that the visualization can be reconstructed accurately in its annotated state. These parameters may include both the mappings applied to the data fields and any user-defined configurations that influenced the visualization's appearance at the time of annotation. Specifically, the stored parameters may include variable mappings, applied filters, axis configurations, color schemes, zoom levels, data point selections, and any field-specific transformations. To provide for precise reconstruction of the annotated visualization, the system, in some embodiments, also records metadata related to the dataset version, unique dataset identifiers, and timestamped mappings that define how each variable in the data asset was represented in the visualization. For instance, if the visualization represented an incidence plot of adverse events filtered by treatment duration and severity, these filter settings, in some embodiments, are stored alongside the mappings that classified each adverse event by type and severity level. When the annotation is accessed for review, the system, in some embodiments, retrieves this stored configuration and applies the original parameters to recreate the visualization dynamically. The system, in some embodiments, programmatically re-applies each filter, mapping, and transformation, restoring the visualization to the exact state in which it was originally annotated. This approach enables reviewers to see the visualization precisely as it appeared when the annotation was made, complete with the data, mappings, and user configurations that provide essential context for accurate issue analysis and resolution.

Mappings are generated, in some embodiments, through a multi-step semi-automated or fully automated process executed on a computing system. In the first step, in some embodiments, data domains and classes are automatically inferred for each raw input dataset. The input data, in some embodiments, is scanned and algorithms search, in some embodiments, for variable names or structures that would be expected for different dataset classes. For example, if the data comes from a CDISC format such as ADaM or SDTM, the algorithm detects that this is the case and uses what is known about the structure of these datasets to determine what class each dataset belongs to. In the case of CDISC datasets, data domains and classes can be ascertained by looking at the file name (e.g. ADaM datasets for adverse events typically have “aeds” found in their file name) and standard variable naming practices. The algorithms, in some embodiments, provide a best prediction of each dataset's domain and class and pass this the second step of the process which is a user interface where the user can confirm or update the choices made by the algorithm. Once the user is satisfied with the data domain and class designations, in some embodiments, an algorithm takes these specifications for each dataset and makes an attempt at automatic determination of default mappings, the automatic determination potentially being performed by machine learning model. The result, in some embodiments, may then be presented back to the user who can modify the mappings or provide mappings for any that could not be inferred.

To facilitate accurate mappings, in some embodiments, the system may provide an interactive interface where users can review, adjust, or manually input mappings as necessary. Users may access the suggested mappings for each data asset and have the option to confirm, reject, or modify these mappings. For fields that are not automatically mapped or are incorrectly assigned, users may manually input or reassign mappings to ensure each data field is correctly integrated into the shared schema. For example, a user could reassign a misclassified “Start_Date” field to align it with the intended “treatment start date” attribute. The computer system may also incorporate a validation and confirmation workflow, which prompts users to confirm adjustments or manually input mappings that diverge from default settings. This validation step, in some embodiments, enhances data integrity within the transformed dataset. Furthermore, all user interactions related to mapping adjustments are logged, with metadata such as user ID, timestamps, and change descriptions recorded to enable traceability and version control.

In some embodiments, user interactions with mappings contribute to a feedback loop within the machine learning model, allowing the model to adjust its predictions based on confirmed or modified mappings. This feedback, in some embodiments, helps the system to refine its automatic mapping accuracy over time, effectively learning from user adjustments and improving the transformation process for future data assets.

Some embodiments may execute an extract transform and load process to ingest raw clinical data from clinical trials into the above-described unified general data model or similar data models.

In some embodiments, the system executes an extract, transform, and load (ETL) process to ingest raw clinical data from clinical trials into the unified general data model or a similar standardized data model. The ETL process begins with the extraction phase, where data assets are retrieved from various sources, including electronic case report forms (CRFs), laboratory systems, or other electronic data capture (EDC) systems. This extraction phase is designed to handle multiple data formats and protocols, supporting both structured and semi-structured data inputs from sources such as SQL databases, XML files, and RESTful APIs. The transformation phase, in some embodiments, applies a series of operations to map the extracted data fields to the shared schema defined by the unified general data model. This transformation, in some embodiments, includes standardizing data types, reformatting fields (e.g., converting date formats), and mapping variables to predefined schema attributes based on clinical trial standards (e.g., CDISC SDTM and ADaM formats). During this phase, any missing values or inconsistencies in the data are addressed through predefined handling rules or imputation techniques, ensuring that the transformed data adheres to the requirements of the unified model. For complex mappings, the system may use machine learning algorithms to automatically classify and align fields based on learned patterns in data attributes. In the loading phase, the transformed data is imported into the system's database, represented according to the unified data model. This step involves assigning unique identifiers to each dataset and storing metadata such as the source of the data, ingestion timestamp, and any relevant transformation parameters. The loaded data is stored in a format optimized for analysis and visualization, allowing users to interact with standardized clinical data seamlessly.

In some embodiments, this may include obtaining a plurality of records from a clinical trial from a given user with the above-described software as a service server system. In some cases, that server system may maintain separate tenant accounts for a plurality of different tenants hosting or analyzing data related to a plurality of different clinical trials. Some embodiments may maintain roles and permissions associated with user accounts under those tenant accounts by which access is selectively granted to different clinical trial data and transformations thereto.

In some embodiments, the clinical trial data in the raw format is obtained as electronic case report forms or paper documents. In some cases, paper documents may be scanned and processed with optical character recognition for data entry. Different tenants may have exclusive access to only their clinical trials, with some tenants having multiple clinical trials with data hosted, and formats for different raw clinical trial data being different among tenants and, in some cases, among clinical trials for a given tenant.

Some embodiments may then receive or otherwise obtain an identifier of the clinical trial corresponding to the format of the case report forms. Some embodiments may then look up a previously associated classes and subclasses (e.g., a class hierarchy) using the techniques described above to transform the raw clinical trial data into the data format described above or similar data format. This data may be stored by the model as previous transformation data and be used to provide an expedited transformation of raw data assets. In some embodiments, the various above-described classes may be instantiated and configured differently at different levels for different case report formats or different identified clinical trials.

In some cases, some of the classes may be shared across some or all of the clinical trials, while other classes, such as certain subclasses, may be specific to subsets of a population of clinical trials. And some subclasses may be specific to just one clinical trial. In some cases, a transformation may be defined by identifying or otherwise specifying this set of classes and subclasses to be instantiated.

Some embodiments may then instantiate the identified or otherwise specified subclasses and classes and apply a transformation method associated with those classes to the corresponding fields in the raw clinical trial data. In some cases, the methods may include an identifier of a field name in a namespace of the respective clinical trial (e.g., a regex, or keyword), with different clinical trials having different namespaces. In some cases, the classes may also identify starting formats for data in the raw clinical trial data, such as date formats, address formats, name formats, and the like, and output formats for the same, with some or all clinical trial data transformations using the same output format, regardless of input format. In some cases, the instantiated classes may include methods that transform the resulting raw input data into the data format described above or other similar formats. In some cases, some or all of the plurality of different clinical trials may have raw clinical trial data that can be transformed by the present system and some embodiments into the unified data format.

Leveraging the properties of inheritance afforded by object-oriented programming languages may reduce computational complexity and developer effort when specifying these transformations, as aspects of transformations that are common to all clinical studies may be specified at higher levels of the hierarchy of classes, while more highly variable aspects may be specified at lower levels of the hierarchy, thereby affording reuse of code and data, while still accommodating a relatively diverse set of formats among the input raw clinical trial data. Some embodiments may implement similar approaches by applying polymorphism to methods.

An inherency structure, while enhancing modularity and code reuse, can introduce increased algorithmic complexity and memory usage, which are performance concerns in computer science. Each additional layer in the hierarchy may require the system to traverse multiple levels to resolve method calls, adding computational overhead and potentially increasing the time complexity of certain operations, particularly in deep or complex hierarchies. Furthermore, as each subclass inherits properties and methods from parent classes, the cumulative memory footprint grows with each instance, as objects retain inherited attributes in addition to subclass-specific fields. This expanded memory usage, combined with increased computational demands, can impact overall performance, especially when scaling the system to handle extensive clinical trial data across multiple, specialized dataset types.

In some embodiments, determining inheritance within hierarchical classes may involve traversing a directed acyclic graph (DAG) or tree structure that represents the inheritance relationships among classes. The computational complexity of resolving inheritance may depend on the depth and breadth of the hierarchy. For example, in a tree structure where each node represents a class and edges denote parent-child relationships, a depth-first or breadth-first traversal may be employed to identify all ancestors or descendants of a given class. The complexity of such traversal may be O(n) in some embodiments, where n represents the total number of nodes (classes) in the hierarchy. However, in scenarios where multiple inheritance is allowed, the structure may resemble a more general DAG, and resolving inheritance chains may involve identifying cycles or redundant paths, which may require additional operations. In such cases, algorithms to detect and eliminate redundant edges may have complexity proportional to O(e+n), where e is the number of edges in the DAG. Additionally, caching or memoization may be incorporated in some embodiments to reduce repeated computations, which may further influence the effective complexity of inheritance determination. This may slow operations in ways that can be undesirable in some use cases, which is not to suggest that embodiments that suffer from this issue are disclaimed or disavowed.

To mitigate these issues, some embodiments may precompute (e.g., before used in transformations) inheritance properties of dataset classes. This may involve analyzing the hierarchical relationships among classes and storing a flattened representation that encodes these relationships for direct access. This precomputation may traverse (e.g., depth first or breadth first recursive traversal) the class hierarchy, identifying all ancestor and descendant relationships for each class, and generating a lookup table or matrix where entries indicate whether a direct or indirect inheritance relationship exists between two classes. For example, a binary matrix representation may store a “1” at position (i, j) if class i inherits from class j, directly or transitively. This preprocessing step may employ algorithms with a complexity of O(n\hat{ }2) for n classes to populate the matrix, particularly in dense hierarchies. Once precomputed, operations that require checking inheritance relationships may be performed in O(1) time by querying the precomputed structure. Flattening relationships may also involve aggregating attributes or methods associated with ancestor classes into a single representation for each class, allowing application-time operations to access these consolidated properties without needing further traversal, thereby expediting subsequent transformations based on the dataset classes. In some embodiments, additional optimizations such as indexing or partitioning of the lookup data may be used to reduct memory consumption while maintaining efficient access.

In some embodiments, to optimize (e.g., to improve or attain a global optimum) method lookup times in an inheritance structure, the system may implement selective method caching and lookup optimization. This approach may involve caching frequently accessed methods, particularly those located at higher levels in the inheritance hierarchy. By storing commonly used methods in memory, the system can bypass redundant traversal of multiple inheritance layers when these methods are invoked. For instance, a commonly inherited method for data transformation or mapping application could be cached, allowing the system to retrieve the method directly from memory instead of initiating a full traversal up the hierarchy. This method of caching minimizes traversal time across inheritance levels, enhancing performance in environments where method calls occur frequently or real-time processing is required. Some embodiments may cache mappings of the dataset classes in a hash map, such as a nested hash map, and transformations may be expedited by indexing into the precomputed mappings with the hash map.

In some embodiments, precomputing mappings from a hierarchy of classes may involve generating and caching associations between classes and their relevant properties, such as ancestor relationships, descendant relationships, or aggregated attributes, within a hash map. The precomputation process may traverse the hierarchy to derive these mappings, which may include paths from each class to its ancestors or summaries of inherited attributes and methods. The resulting mappings may be stored in the hash map, where each class identifier serves as a key and the corresponding precomputed data serves as the value. By leveraging the O(1) average-case access time of hash maps, subsequent lookups of inheritance-related information for a given class may be significantly expedited compared to traversing the hierarchy directly, which may involve O(n) operations for a hierarchy with n nodes in the worst case. This cached approach allows multiple accesses of the same mapping during transformation operations to bypass redundant traversals, reducing computational overhead. In some embodiments, the hash map may be designed to accommodate dynamic updates to the hierarchy, such as when new classes are added, by selectively invalidating and recomputing affected mappings, maintaining efficiency while ensuring correctness.

In some embodiments, a hash map may function to access precomputed mappings by employing a hash function to map keys to specific locations in an underlying data storage structure, such as an array or similar construct. Each key, which may represent an identifier for a class or an entity, is processed through the hash function to compute a hash value. This hash value may correspond to an index in the data storage structure where the associated value is stored. For example, a key representing a class may be passed to the hash function, which produces an integer value. This integer value may be used to directly access the corresponding entry in the array-like storage, resulting in O(1) average-case access time.

This or other forms of caching may be applied in some embodiments. The caching process may be initiated after a method or transformation rule is first executed within the hierarchy. Once a method is invoked and applied to a data asset, the system stores this method in a caching layer, typically organized as a key-value store in memory or a caching database. Each cached item is associated with a unique key based on its specific attributes—such as the data type, transformation parameters, and hierarchical location—enabling quick access when similar data assets require the same transformation. In this way, when a method is cached, it becomes accessible to all subclasses that inherit from the same base class, enabling shared use across the hierarchy without redundant lookups. In subsequent transformations, the system first checks the cache for any relevant methods or rules before initiating a lookup in the inheritance chain. If a cached transformation method or rule is available, the system may apply it directly to the data asset, reducing the time complexity of lookups from O(n), where n represents the inheritance depth, to approximately O(1) for cached items. Additionally, the system includes cache invalidation mechanisms to refresh cached items when changes are made to a method or rule in the base class, ensuring that cached methods remain accurate and consistent. This approach optimizes both performance and resource efficiency, making the inheritance structure scalable even in high-demand environments such as real-time clinical trial data processing.

Some embodiments may employ polymorphic interfaces and delegation in place of direct inheritance for specific functionalities. Rather than binding subclasses directly to inherited methods, the system defines polymorphic interfaces that dataset classes can implement according to their unique requirements. For example, shared functionalities such as data transformation or data mapping can be defined in interfaces that various dataset classes implement independently, allowing each class to execute these functions as needed without creating dependencies on inherited base-class methods. In addition, the system can use delegation, where an object within the class, rather than the class itself, is responsible for carrying out specific tasks. For example, an adverse event dataset class could delegate data validation functions to a separate validation module, enabling the dataset class to focus solely on managing adverse event records. This approach supports flexible functionality across dataset types without adding layers to the inheritance hierarchy, as classes can access only the specific behaviors they require, rather than inheriting additional properties that may not be relevant.

In some embodiments, the transformation may include parsing the raw clinical trial data, detecting field names and associated values, and calling the corresponding method to transform those values associated with the corresponding classes for that transformation. Some embodiments may also verify that data is not in error, e.g., checking for ages more than 130 years old, checking for dates in the future, checking for birthdays on the 31^{st} in months that only have 30 days, etc. Erroneous entries may be flagged, e.g., with an issue tracking mechanism like those described below, for further investigation.

Some embodiments include one or more of the following features, which may be implemented server-side in a SaaS distributed architecture communicating with a client computing device or as a monolithic application executing locally on a client computing device:

Digital Entities in Clinical Trials: In a clinical trial setting, the digital entities (or other data assets) mentioned may include patient data, drug dosage and response information, diagnostic reports, statistical analyses, graphical visualizations of trends and responses, and summary reports of each phase of the trial. In some embodiments, each digital entity is associated with metadata that tracks its origin, date of capture, data source, and any transformations applied to it during ingestion or analysis. The system may store this metadata alongside the digital entity, allowing for traceability and auditability. When deployed as SaaS, these digital entities may be synchronized across distributed database servers to ensure data consistency and support real-time access from client devices. In a monolithic configuration, the digital entities may be stored locally, enabling low-latency access, particularly useful in environments where network connectivity may be limited or where data security requires on-premises storage.

Issue Tracking Mechanism: As clinical trials progress, various issues may arise within the data, such as inconsistencies in data entries, unexpected patterns, or anomalies in analysis results. The system's issue tracking mechanism is designed to capture, document, and manage these issues in a structured and traceable manner. In some embodiments, the issue tracking mechanism allows users to flag specific anomalies and link them directly to the dataset, data entry, or visualization where the issue was detected. This linking capability provides essential context for issue resolution, ensuring that reviewers and analysts can efficiently investigate and address the root cause. Each issue recorded in the system is accompanied by metadata, including the date and time of the issue's creation, the user ID of the individual who flagged it, and any parameters that were active at the time (e.g., data filters, visualization settings). The system may also log the data version in use, ensuring that reviewers can trace back to the exact data state when the issue was identified. The system logs all actions taken to address each issue, creating an audit trail that documents each step of the resolution process. For example, if an issue leads to the reclassification of data or an adjustment in data handling procedures, these actions are documented within the issue tracking mechanism. This provides transparency and traceability, ensuring compliance with regulatory requirements and facilitating a robust quality control process throughout the trial lifecycle.

Notifications to Stakeholders: Notifications to Stakeholders: Timely notifications are critical in clinical trials to ensure prompt awareness and action on data integrity concerns, procedural updates, or other trial-related issues. In some embodiments, the system includes an automated notification mechanism that monitors for specific conditions or events and sends alerts to relevant stakeholders, such as researchers, data analysts, clinicians, and regulatory bodies. These notifications can be triggered by various events, such as the detection of data inconsistencies, the flagging of critical issues, completion of data transformations, or updates to trial results. The system is designed to support multiple notification channels to ensure that stakeholders are reached effectively. Notifications may be sent through SMS (short message service), email, or other auxiliary channels such as push notifications within a mobile app or desktop alerts on the platform's user interface. These channels allow users to configure their preferred notification settings based on their role, urgency level, and type of alert. For instance, high-priority notifications related to data integrity concerns or urgent trial updates may be sent via SMS to ensure immediate attention, while lower-priority updates may be routed to email or displayed as in-app notifications.

Asset Versioning Mechanism: In the dynamic environment of clinical trials, data might be updated or corrected frequently. Some embodiments have an asset versioning mechanism that ensures that every iteration or other change (or at least some iterations satisfying various criteria for tracking) made to any digital asset (like a patient's medical record or an analysis graph) is recorded. This is expected to be helpful in some cases, e.g., if data needs to be reviewed or if concerns arise regarding its validity. For instance, if initial data indicated a positive drug response, but later corrections altered that view, researchers can trace back to see what changed and why in some use cases.

Search Module: In clinical trials, where thousands of data points and multiple issues may arise across patient records, adverse events, and trial phases, having a robust search and logging module is essential. This module provides comprehensive search functionality, enabling users to efficiently locate specific data assets, review patient histories, track issue resolution progress, or retrieve previous versions of analyses. The search functionality is designed to support complex queries, enabling users to filter results based on multiple criteria such as patient ID, event type, date range, data field values, or issue status. The system may support advanced search capabilities through a combination of structured querying options and free-text search, allowing users to refine their queries for precise data retrieval. For instance, a researcher could use structured search to locate records for all patients within a specific age range who reported a particular adverse event. For free-text search, the system may use indexing techniques—such as inverted indices for keyword searching and full-text indexing for longer text fields—to quickly retrieve records that match keywords or phrases in unstructured fields, such as clinician notes or patient-reported outcomes. To handle multi-dimensional data, the system may incorporate faceted search functionality, where search results are grouped and categorized by key attributes, such as trial phase, treatment group, or data type. This allows users to further refine their results by selecting specific facets, such as viewing only those records relevant to a particular adverse event or a given study cohort.

Logging Module: The logging functionality complements search by maintaining a detailed, time-stamped record of all data interactions and modifications. The logging component captures and stores metadata for each interaction, including user ID, action type (e.g., view, edit, delete), and data asset ID. This creates an audit trail that documents the entire history of data interactions, which is essential for both regulatory compliance and data integrity. The audit trail ensures that any changes to patient records, data transformations, or issue statuses are fully traceable, allowing users to review the evolution of a data asset or issue over time. The search and logging module may support saved queries and search history functionality, saving frequently used search parameters and access recent queries. This feature enhances workflow efficiency by allowing users to quickly reapply search criteria without re-entering them. Additionally, in scenarios where specific search results need to be shared or retained for future analysis, the module may allow users to export search results or save them within a reporting framework.

Ensuring Reliability and Authenticity: To uphold data integrity and foster trust, some embodiments are designed to place clinical trial data on a robust foundation of transparency and traceability. These embodiments may incorporate multiple layers of validation, logging, and data tracking to ensure that every interaction with clinical trial data is verifiable, allowing stakeholders to confidently rely on data provenance. Each data asset within the system may be accompanied by detailed provenance metadata, which records the data's origin, entry method, source, and timestamp. For instance, patient records ingested from Electronic Data Capture (EDC) systems, laboratory tests, or diagnostic reports may be tagged with metadata such as acquisition date, device or software source, and clinical site identifiers. This metadata is continuously logged and updated with each interaction, creating a complete lineage record for tracking the data's source.

The system may also maintain an immutable audit trail, capturing every modification, view, and interaction with each data asset. This audit trail includes time-stamped logs of actions, user IDs, and descriptions of changes, ensuring an unbroken chain of custody for each piece of data. To protect data authenticity further, some embodiments employ cryptographic techniques, such as cryptographic hash pointers, making unauthorized modifications immediately detectable. Data assets are stored in hash-linked structures, where each record is associated with a cryptographic hash that links to the previous version.

Role-based access control policies may further enhance reliability by limiting data access and modifications based on user roles. Researchers, clinicians, data analysts, and regulatory auditors are assigned access levels aligned with their responsibilities, restricting the ability to view, edit, or approve specific data assets. These permissions may be embedded within the system and recorded in the audit log, providing accountability and reducing the risk of unauthorized changes. In addition to access control, the system may incorporate periodic data verification procedures to validate the accuracy and completeness of data assets. Consistency checks, such as cross-referencing adverse event records with clinical protocols and recalculating statistical outputs, ensure data remains accurate and error-free.

Deploying some embodiments within the environment of clinical trials is expected to offer an enhanced layer of reliability, transparency, and accountability. Given the stakes involved in clinical research—from patient safety to scientific integrity—having a scientifically designed framework like this can be helpful in some cases.

### SAAS Architecture

Some embodiments may be implemented in SaaS environment. In some embodiments, the physical architecture of the system may utilize cloud services, such as those provided by AWS (Amazon Web Services™), Google Cloud™, Azure™, or other similar providers. This may allow for scalability, availability, and heightened security. These cloud services may be composed of a combination of servers including web servers, application servers, and database servers. In some configurations, load balancers may be employed to distribute incoming application traffic across multiple target servers or systems. Storage systems may encompass multi-tiered storage options, from fast-access solid state drives (SSDs) for frequently accessed data to hard drives for archival storage. In certain embodiments, the system may further comprise a backup and disaster recovery subsystem, wherein data is backed up regularly and stored in geographically separate locations to ensure data integrity and availability.

Network security in some embodiments can be achieved using firewalls to protect the internal network from external threats. The invention may operate within a virtual private cloud (VPC) to maintain data isolation and security. Data in transit may be encrypted using protocols such as transport layer security (TLS), while data at rest may also undergo encryption protocols, such as symmetric or asymmetric encryption protocols.

Monitoring and logging tools, including but not limited to solutions like Amazon CloudWatch™, ELK Stack, Datadog™, or their equivalents, may be integrated for real-time monitoring, logging, and alert generation.

The logical architecture of the system may, in some embodiments, feature a user interface (UI) layer. This layer may comprise a web interface for researchers, scientists, and clinicians to input and view clinical trial data. In other embodiments, a mobile interface can be provided to deliver functionalities on mobile devices. Furthermore, the system may offer API (application program interface) endpoints, facilitating efforts by third-party applications or systems to communicate with the platform.

In some configurations, an application layer may be present. This layer may encompass authentication and authorization mechanisms, potentially utilizing protocols such as OAuth, SSO (single sign on), or others. Business logic may define and process all rules, transformations, computations, and validations related to clinical trial data. Data analytics and reporting capabilities may be integrated, offering advanced analytics, visualization, and reporting features.

The data access layer, present in certain embodiments, can utilize a database management system (DBMS). This could be a relational database such as PostgreSQL or MySQL, or a non-relational database like MongoDB or Cassandra, among others. Object-Relational Mapping (ORM) tools, like Hibernate, Sequelize, or their equivalents, may be employed to manage and facilitate database operations.

The data layer of the system may store clinical trial data, which can include patient information, results, medications, dosages, side effects, and other relevant data. In some configurations, audit logs might track changes to this data to ensure traceability and accountability. Metadata about the data, which may encompass details about its origin, time of capture, the device used, and other related attributes, can also be stored.

Integration capabilities can be present in some embodiments, allowing for third-party integrations. These integrations can facilitate connections with other clinical systems, labs, EHRs (Electronic Health Records), or other relevant systems. Data import and export tools and interfaces may be provided to facilitate the movement of data to and from the system.

A security and compliance layer may be incorporated in certain embodiments. This layer™ can ensure compliance with standards such as HIPAA (Health Insurance Portability and Accountability Act) and other relevant regulations. Role-Based Access Control (RBAC) may be used to define what users can view or modify based on their specific roles. In some instances, data masking and anonymization features can be added, ensuring that sensitive data remains non- identifiable.

An operations and maintenance layer™ may be present, facilitating automated testing, with tools and scripts designed to validate functionality, security, and performance. Continuous Integration/Continuous Deployment (CI/CD) pipelines may be employed to automate the testing, building, and deployment of software updates and improvements.

Furthermore, embodiments may include features such as real-time collaboration tools, advanced AI-driven analytics modules, voice recognition for data input, virtual assistants for guiding users, predictive modeling for trial outcomes, and automated alerts for anomaly detection in trial data.

### Tamper-Evident Data

Some embodiments may include techniques designed to render changes and updates tamper evident to provide greater assurances of data integrity. In some embodiments, a system and method are provided for rendering data assets in a data tracking system tamper-evident, leveraging cryptographic techniques combined with strategies for verifying the data's state over time.

In one embodiment, the system may utilize a data structure like Directed Acyclic Graphs (DAGs) with Cryptographic Hash Pointers, like a blockchain, where each block may also contain a hash pointer, which may point to the previous block in the sequence. This hash pointer may be a cryptographic hash of the entire content of the aforementioned previous block. Any slight alteration to the content could drastically change this hash, which may subsequently indicate potential tampering. The data in a block might include, but is not limited to, the above-described data assets, metadata, or any other relevant information.

In some instances, the system may incorporate the principles of cryptographic accumulators. These accumulators have the capability to consolidate multiple values into a single value. Given an accumulator and a specific value, one might ascertain whether the said value was incorporated into it without disclosing other amalgamated values. An exemplary approach to this may be the employment of Merkle Trees. A Merkle Tree, in certain embodiments, may start with individual data points (often referred to as “leaves”), and these leaves may be combined in pairs, hashed together, eventually leading to a single top hash (or “root”).

Furthermore, some embodiments may choose to leverage cryptographic signatures to enhance the tamper-evident properties of the system. Digital signatures could offer a means to verify both the integrity and authenticity of data assets. Public-key infrastructure (PKI) might be utilized in such instances. An asset could be signed using the private key of a trusted entity, and anyone in possession of the corresponding public key may then validate the data's authenticity. The digital signature process might include generating a signature based on the data asset and the private key and subsequently verifying the signature using the data asset and the corresponding public key. Data assets, edits thereto, and comments thereon may be cryptographically signed by those inputting the relevant information.

In another embodiment, the system might benefit from timestamping. Trusted timestamping services could vouch for a particular state of the data at a specific point in time. If the data undergoes any modifications, the original timestamp might not correspond to the altered state, thus highlighting discrepancies. In addition, the tamper-evident properties might be bolstered by incorporating redundancy and replication mechanisms. Distributing multiple replicas of the data across diverse storage mediums or providers—spanning different geographical locations, technological platforms, or cloud providers—may make any clandestine modifications to the data more discernible.

A developer, when implementing the above system for data integrity, may include features like the following: 1. Advanced cryptographic algorithms for improved security and performance; 2. Layered security protocols, where multiple cryptographic techniques are applied in tandem; 3. Integration with secure hardware modules for enhanced protection of cryptographic keys; 4. Real-time monitoring and alert systems that notify stakeholders of potential data anomalies or suspected tampering; 5. Advanced data recovery mechanisms to restore data to its last known authentic state in the event of tampering; and 6. Integration with distributed ledger technologies beyond blockchains.

### Search Techniques

Some embodiments may implement search over data assets using various Information Retrieval (IR) models. In some embodiments, the search functionality may incorporate a Classical IR model. Within such embodiments, a user might input Boolean expressions, for example, “Apple AND Orange” or “Banana NOT Grapes.” In these instances, the system may search for documents or data entries that meet the specified Boolean expression criteria. Additionally, documents and user queries can be represented as vectors. Such a system may have an integrated search bar where users provide their query. Behind the scenes, the application may transform both documents and queries into vectors. By comparing these vectors, the system can determine similarity and possibly rank search results based on this similarity. In some embodiments, options for representation may include Binary in Boolean Vector Space Model (VSM) or Weighted in Non-binary VSM. Moreover, some embodiments may treat documents (such as data asserts) as distributions of terms, wherein user searches result in the application comparing the similarity of term distributions between the query and documents. The system may then display results based on calculations such as entropy or the probable utility of the document. In other embodiments, a ranking algorithm can be integrated, ranking documents based on their probability of relevance to a user's search query.

In other embodiments, the system may use a Non-Classical IR model. Such a model may be based on propositional logic, allowing users to create complex queries. The system may then interpret these logic-based queries to obtain relevant documents or other data assets. In some situations, the system may consider the user's context or situation to enhance search result relevance. This can be integrated with user profiles or behavior analytics.

Alternative IR models may also be employed. In some embodiments, the system might group similar data assets into clusters. Upon a user search, the system may identify the most relevant cluster(s) and retrieve data assets from those clusters. Latent Semantic Indexing (LSI) may be used in other embodiments to analyze the relationships between the terms in documents, allowing the system to identify hidden semantic structures and provide search results based not only on exact term matches but also the semantic meaning of the query. Additionally, some embodiments might use the Fuzzy Set model, especially beneficial when user queries are imprecise or when documents contain ambiguities. In such cases, the system might consider approximate matches to provide results aligning with the user's intent. Furthermore, a Generalized Vector Space Model can be employed in some embodiments, enhancing the vector space model by considering additional factors or dimensions when representing documents and queries as vectors.

Some embodiments may include features such as voice search capabilities, natural language processing to better understand user intent, multi-language support for global applications, or integration with other third-party applications and data sources to enhance search depth and breadth. Machine learning algorithms may be incorporated to continuously learn and improve from user behavior and feedback. The system may also include a preference for commonly accessed documents, giving them priority in search results. Visualization tools might be added to represent search results in graphical or chart formats.

In some embodiments, the system may pre-index data in advance to expedite query responses, enabling rapid access to specific data points and minimizing latency during data retrieval. This indexing process may involve creating structured indices on frequently queried fields, such as unique subject identifiers, event dates, adverse event classifications, and treatment durations. By organizing data into these searchable indices, the system may reduce the time required to locate and retrieve specific records during analysis or visualization tasks.

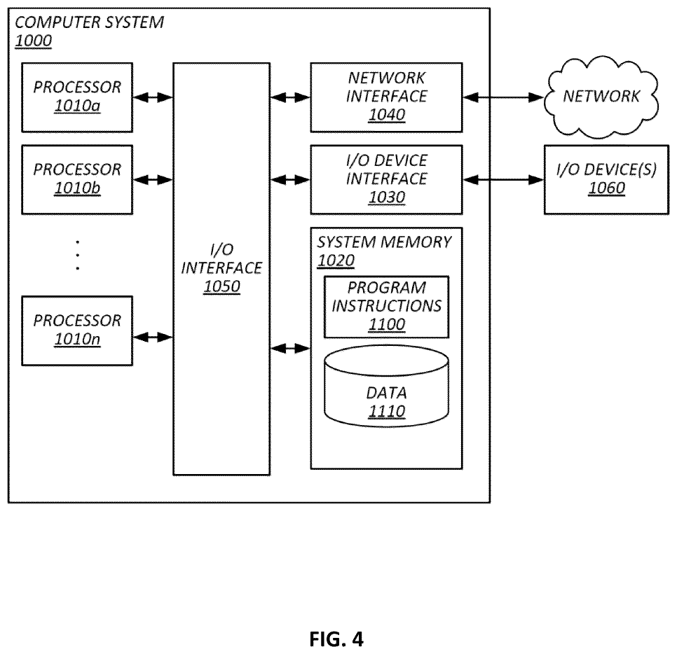

Some embodiments may implement the present techniques on a remote server system that interfaces with user interfaces on various client computing devices (like web browsers or special purpose applications executing thereon) via a network, like the internet. In some embodiments, the operations herein may be executed on the server system, such as one implemented with one or more of the computing devices in FIG. 4.

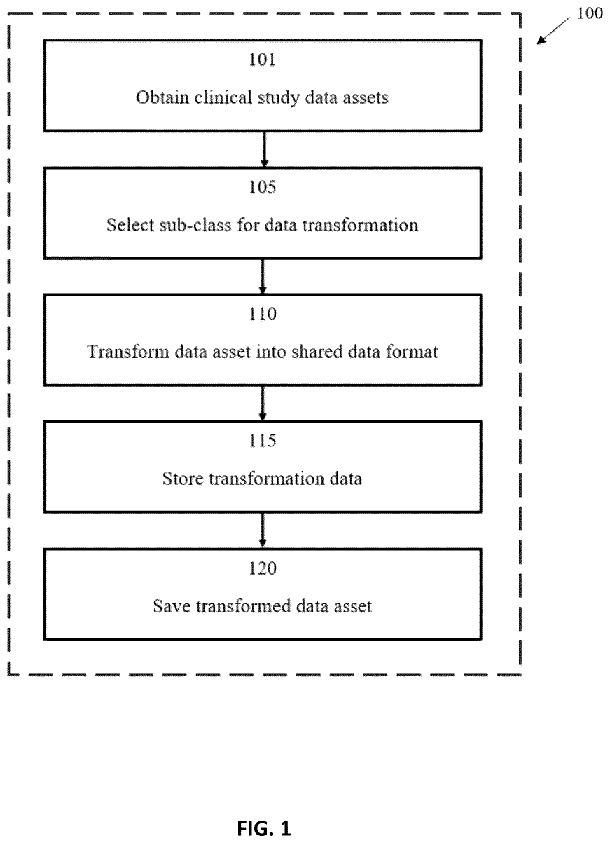

FIG. 1 illustrates a block diagram detailing the method 100 for processing clinical trial data assets into a shared data format. The method begins, in some embodiments, by obtaining clinical study assets 101, followed by selecting an appropriate subclass for data transformation 105, transforming the data into the shared data format 110, storing the transformation data associated with the transformation process 115, and saving the resulting transformed data asset 120. This sequence, in some embodiments, helps the system to standardize data assets from varying initial formats into a unified format that supports consistent analysis and visualization.

The method 100 begins, in some embodiments, by obtaining a plurality of raw clinical data asset 101, which may be formatted in diverse data schemas or a single, consistent schema. These formats may include any of the previously described standards, such as SDTM or ADaM, or other structures tailored to support the storage of clinical study data. In addition, the raw data assets may also encompass non-standard formats from diverse data sources, allowing the method to accommodate and standardize a range of data assets beyond those strictly related to clinical trials. In some cases, data may be in the form of image PDFs or image TIFFs, and an optical character recognition pre-processing step may be applied.

Once the data is obtained, the method 100 proceeds to select an appropriate subclass for data transformation 105. This selection process may be initiated by either a user or the computer system (e.g., a server system). In cases where the user performs the selection, the system may display a prompt with available subclasses. The user can then choose a subclass for transformation, with the option to apply this selection to multiple untransformed data assets simultaneously or to designate a unique subclass for each data asset. If the subclass selection step 105 is automated by the computer system, the system may employ a predefined algorithm or predictive analysis model configured to choose a subclass based on the specific characteristics of each data asset. This selection step 105 may involve clustering data assets with similar attributes and applying a common subclass to those clusters, or it may involve individualized subclass assignments for each data asset based on the asset's specific schema or format.

After selecting a subclass 105, in some embodiments, the method 100 proceeds to transform each data asset into a shared data format 110. Transformation of the data asset into a shared data format 110, in some embodiments, involves associating the selected subclass's mappings with variables within the raw data asset. Each attribute or variable in the raw data asset may be mapped to its corresponding attribute in the shared data format, the transformation being performed based on instructions related to mappings contained within the selected subclass in some cases. The transformed data asset, in some embodiments, is generated based on these mappings. This is expected to help with compatibility with the unified model and providing consistent data analysis across various clinical trials.

Following transformation into the shared data format 110, in some embodiments, the method 100 stores metadata associated with the transformation process 115. This transformation data may include details about the specific subclass selected, the format and schema of the original data asset prior to transformation, timestamps for when the transformation occurred, and indicators confirming transformation success or noting identified issues. Additional metadata may capture transformation parameters, such as any specific mappings applied or user-defined overrides, to provide a complete record of the transformation.

After storing transformation data associated with the transformation process 115, the method 100, in some embodiments, saves the transformed data asset 120, the data asset now presented in the shared data format. At this stage, the transformed data asset is optimized for further analytical processes and visualization, which may help with integration with other standardized data assets within the platform.

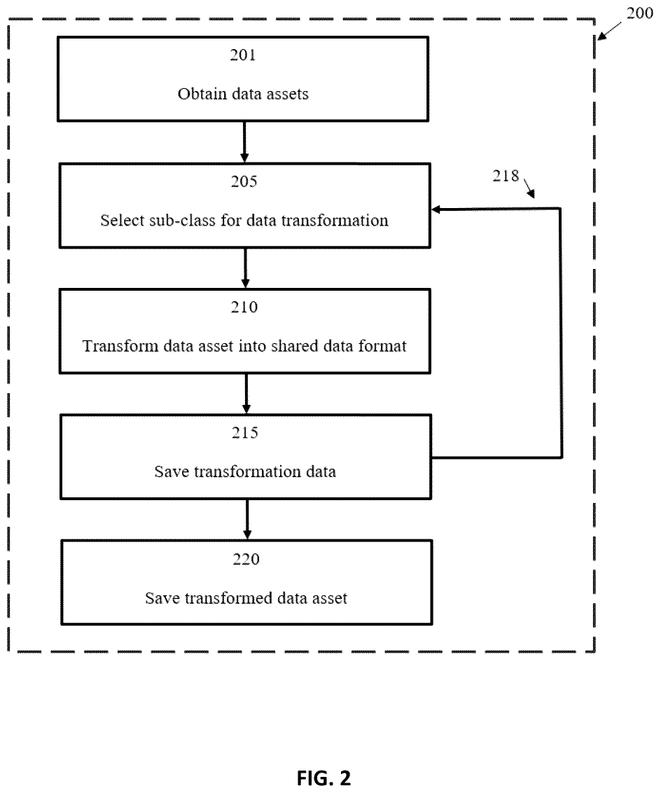

FIG. 2 details a method with a feedback loop 218, wherein transformation data saved after each data transformation 210 is utilized by the computer system to improve future processing steps. The description of corresponding steps in FIG. 1 applies here. This transformation data, in some embodiments, is stored 215 by the system and includes metadata relevant to performed transformations such as the selected subclass, transformation parameters, timestamps, success indicators, or issue indications. Once incorporated into the feedback loop 218, this stored transformation data, in some embodiments, is analyzed and used to inform subsequent subclass selections for data transformation 205.

When the feedback loop 218 is active, the method 200, in some embodiments, learns from previous transformation results (e.g., continuously, as a batch process, periodically, or intermittently), potentially providing for more efficient and accurate subclass selection in future processes. By referencing saved transformation data, in some embodiments, the system can detect patterns in data assets that share similar formats, schemas, or attribute structures. For example, if a certain subclass consistently produces high-quality transformations for a particular format, the system can prioritize this subclass for similar data assets, reducing the computational cost of evaluating all possible subclasses. This may minimize (or reduce) redundant computations, as the system bypasses the need to perform extensive analysis on known data structures, instead relying on proven transformation paths.

The feedback loop may enhance user interaction by suggesting optimal subclasses based on prior transformation successes. In the instance that selection of a subclass is being performed by a user, the system may present the user with suggested subclasses, ranked by historical performance metrics, which may include transformation speed, error rate, or accuracy in producing the shared data format. These suggestions, in some embodiments, streamline user decisions and reduce the risk of errors, ensuring that transformations align with system-optimized recommendations.

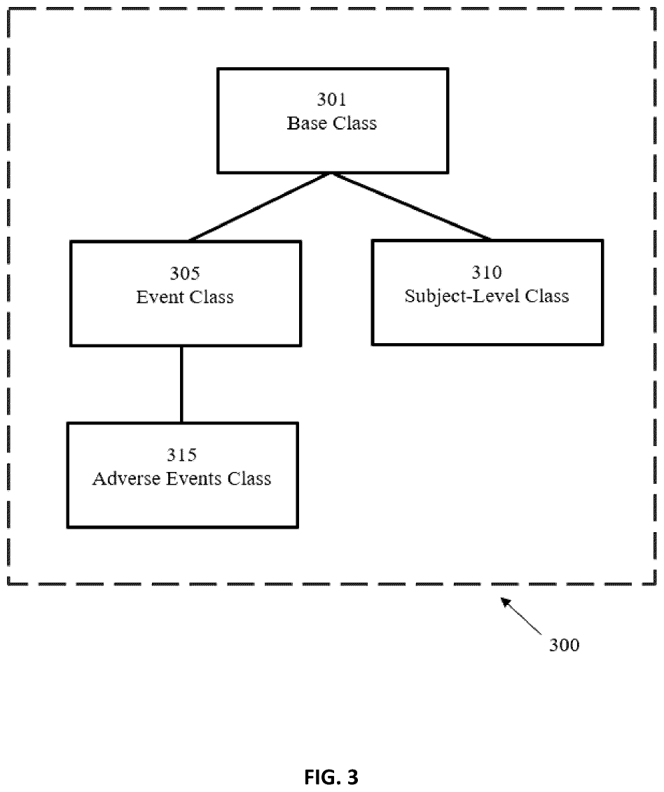

FIG. 3 illustrates an example data structure 300 of the unified data model, which may include mappings designed to standardize and organize clinical trial data for consistent analysis and visualization. The data structure 300 is hierarchically organized, beginning with a base class 301 from which all other classes inherit. The base class 301, in this example, defines attributes and mappings that are shared across to the structure, providing a foundation for more specialized data handling within the shared format, and avoiding the need to re-specify those features in each dataset class that inherits from the base class 301, directly or indirectly through other dataset classes. These attributes may include identifiers, such as a unique subject ID (e.g., a global unique ID, a ID that is unique within a trial, a ID that is unique within a session or project, or an ID that is unique across all data ingested by the system), and metadata fields that are relevant across various data types.

The data structure 300, in some embodiments, includes an event class 305 and a subject-level class 310, both of which inherit from the base class 301. The event class 305, in some embodiments, is tailored for managing data related to events occurring during the clinical trial, such as medical interventions, observations, or milestones in the study timeline. By inheriting from the base class 301, the event class 305, in some embodiments, includes its mappings (e.g., subject identifiers) while introducing additional event-specific attributes, such as start and end times, event descriptions, and event classifications. These attributes help the event class 305 to accurately represent time-sensitive or recurring occurrences within the trial, with mappings that link events to specific subjects or study phases.

The subject-level class 310, in some embodiments, also inherits from the base class 301 and is primarily responsible for organizing data on an individual subject level, consolidating information relevant to each participant in a single structure. This class, in some embodiments, may contain subject-specific mappings such as demographic details, primary treatment variables, baseline health indicators, and longitudinal metrics, such as compliance and adherence records. The subject-level class 310, in some embodiments, provides for a comprehensive view of each participant's profile, linking demographic and treatment data across other classes.

The data structure 300 may also include an adverse events class 315, which inherits from the event class 305, allowing it to manage a subset of events classified specifically as adverse events. The adverse events class 315, in some embodiments, includes all the attributes of the event class, while adding mappings unique to adverse events, such as severity level, treatment emergent status, causality assessment, or any regulatory reporting requirements. The adverse events class 315, in some embodiments, allows for a detailed representation of adverse events, providing for more targeted analyses, such as evaluating the frequency and severity of specific adverse events across study populations or tracking treatment-related side effects.

Some embodiments may apply the techniques described in U.S. Provisional Patent Application 63/614,516, filed 22 Dec. 2023, by the same Applicant, titled DATA INTEGRITY FOR CLINICAL STUDIES, the contents of which are incorporated by reference in their entirety. The incorporated material may be used, for example, to provide for the integrity and traceability of data in clinical studies, leveraging advanced computer-based solutions to address the inefficiencies and risks of manual methods. This may include implementing an automated process for managing digital assets by linking updated versions to prior versions with associated reasons for changes, tracking issues, and notifying relevant stakeholders. Some embodiments may provide search capabilities for retrieving information on clinical data, asset versioning to maintain a history of modifications, and issue tracking mechanisms that connect specific issues to their corresponding data assets. Some such embodiments maybe designed for deployment in flexible environments, such as client devices or cloud-based architectures, with enhanced security protocols and compliance measures to meet industry standards like HIPAA. The system, in some embodiments, as described, may integrate cryptographic techniques, such as hash pointers and digital signatures, to provide for tamper-evident data storage, alongside capabilities for real-time notifications, search functionalities, and automated workflows that support transparency, reliability, and efficiency in clinical trial data management.

FIG. 4 is a diagram that illustrates an exemplary computing system 1000 in accordance with embodiments of the present technique. A single computing device is shown, but some embodiments of a computer system may include multiple computing devices that communicate over a network, for instance in the course of collectively executing various parts of a distributed application. Various portions of systems and methods described herein may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface May 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to cost constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:
1. A method, comprising: obtaining, with a computer system, clinical trial data in diverse formats and schemas; transforming, with the computer system, the clinical trial data into a unified format and a unified schema to produce transformed clinical trial data, wherein transforming comprises: obtaining a hierarchy of dataset classes, wherein: classes further from a root dataset class in the hierarchy inherit from dataset classes closer to the root dataset class in the hierarchy, and the dataset classes map a first set of variables in input data to concepts used in data analysis, a second set of variables in input data to concepts used in visualization, and a third set of variables in input data to concepts used in data operations; the root dataset class maps a patient identifier in input data to a subject identifier in the unified schema, wherein the root dataset class is configured to map different identifiers of the same patient in different input data to the same subject identifier in the unified schema; an event dataset class inherits from the root dataset class and maps input data with diverse formats and schemas characterizing events to event fields in the unified schema, in the unified format; an adverse event dataset class inherits from the event dataset class and maps input data with diverse formats and schemas characterizing adverse to adverse event fields in the unified schema, in the unified format; and a subject level dataset class inherits from the root dataset class and is configured to produce records indexed by the subject identifier in the unified schema, the subject level dataset class being configured to map diverse formats and schemas input data characterizing treatment variables, treatment start dates, and treatment end data into corresponding fields in the unified schema, in the unified format; parsing the clinical trial data into records; determining which dataset classes correspond to each of the records with the hierarchy of dataset classes; and transforming each of the records into the unified format and the unified schema with the corresponding dataset classes in the hierarchy of dataset classes; and storing, with the computer system, the transformed clinical trial data in memory.
2. The method of embodiment 1, wherein at least some of the dataset classes specify visualization, the method further comprising: selecting a plurality of visualization choices as a subset of a set of candidate visualization choices based on visualizations specified by dataset classes that correspond to the records.
3. The method of embodiment 2, wherein the subset comprises visualizations specified by dataset classes from which the dataset classes that correspond to the records inherit in the hierarchy of dataset classes.
4. The method of embodiment 1, comprising precomputing, before the transforming, inheritance properties of the dataset classes to flatten hierarchical relationships, and using the flattened hierarchical relationships to perform the transforming.
5. The method of embodiment 1, comprising: precomputing, before the transforming, mappings from the hierarchy of classes and caching the precomputed mappings with a hash map, wherein multiple accesses of the same mapping during the transforming are expedited by the hash map relative to traversing the hierarchy directly.
6. The method of embodiment 1, wherein the hash map is a nested hash map.
7. The method of embodiment 1, comprising retrieving, during the transforming, dataset classes with a balanced tree index formed before the transforming.
8. The method of embodiment 7, wherein the balanced tree index is an AVL (Adelson-Velsky and Landis) tree, a red-black tree, a B-tree, or a B+ tree.
9. the method of embodiment 1, comprising concurrently determining whether a plurality of dataset classes correspond to a given record among the records formed by parsing the clinical trial data.
10. The method of embodiment 1, comprising concurrently determining whether a plurality of records correspond to a given record among the records formed by parsing the clinical trial data.
11. A method for transforming clinical data into a data model, comprising: obtaining, with a computer system, data assets characterizing a plurality of clinical studies, wherein a first data asset among the data assets is in a first format having a first data schema and a second data asset among the data assets is in a second format having a second data schema; selecting, with the computer system, a first subclass from a hierarchy of classes for the first data asset, wherein the first subclass specifies how to transform the first format and first data schema into a shared data format and a shared data schema; selecting, with the computer system, a second subclass from the hierarchy of classes for the second data asset, wherein the second subclass specifies how to transform the second format and second data schema into the shared data format and the shared data schema, wherein at least one of the shared data format and the shared data schema or both collectively comprise: a base dataset class comprising a mapping relating a subject identifier; an event dataset class inheriting from the base dataset class; and a subject-level dataset class inheriting from the base dataset class, wherein at least some subclasses in the hierarchy of classes inherit from a shared parent class in the hierarchy of classes, and at least some of the subclasses in the hierarchy of classes inherit from a root class in the hierarchy of classes; transforming, with the computer system, the first data asset into the shared data format and the shared data schema using the selected first subclass to produce a version of the first data asset in the shared data format and the shared data schema; transforming, with the computer system, the second data asset into the shared data format and the shared data schema using the selected second subclass to produce a version of the second data asset in the shared data format and the shared data schema; storing, with the computer system, the version of the first data asset in the shared data format and the shared data schema in memory; and storing, with the computer system, the version of the second data asset in the shared data format and the shared data schema in memory.
12. The method of embodiment 11, further comprising: populating, with the computer system, one or more visualizations based on the transformed first data asset, the visualizations corresponding to at least one of the dataset classes; and
receiving, with the computer system, input from a user through an interactive interface to manipulate the one or more visualizations, wherein the input modifies at least one aspect of the visualizations to reflect updated parameters, filters, or analytical views.
13. The method of embodiment 12, wherein the one or more visualizations generated by the computer system comprise at least one of: an incidence plot, a swimmer plot, a univariate numeric summary, or a univariate categorical summary.
14. The method of embodiment 12, wherein the one or more visualizations generated by the computer system comprises: an incidence plot, a swimmer plot, a univariate numeric summary, and a univariate categorical summary.
15. The method of embodiment 11, wherein the event dataset class of the shared data format and data schema further comprises a mapping of a start variable, an end variable, a name variable, and a classification variable.
16. The method of embodiment 11, wherein the shared data format and data schema further comprises: an adverse events dataset class inheriting from the events dataset class, the adverse events dataset class comprising a mapping related to a severity level of an adverse event.
17. The method of embodiment 11, wherein selecting the first subclass further comprises:
preprocessing, with the computer system, the first data asset to identify indicators of a dataset class and domain; and
determining, with the computer system, that a dataset domain and class is to be used based on the indicators identified.
18. The method of embodiment 17, wherein the first data asset is transformed by mapping, with the computer system, the first data asset to a set of default mappings of the determined dataset domain and class.
19. The method of embodiment 17, further comprising:
identifying, within the first transformed data asset, variables corresponding to subject identifiers, event classifications, and subject-level attributes;
creating a subject identifier mapping that links each data record to a subject identifier; and
generating event-specific mappings defining variables related to start and end times, classifications, and descriptions of clinical events.
20. The method of embodiment 19, further comprising:
accessing, with the computer system, stored transformation data;
comparing, with the computer system, the first data format and first data schema of the first data asset with the data formats and data schemas stored in transformation data; and
determining, with the computer system, that the first data asset is to be transformed according to transformation instructions stored in the transformation data based on the comparison.
21. The method of embodiment 11, wherein the first data asset has a different schema and a different format from the second data asset.
22. The method of embodiment 11, wherein the computer system performing the functions of the method is implemented in a cloud-based system architecture, the cloud-based system architecture comprising a plurality of client-facing interface servers and a plurality of database servers.
23. The method of embodiment 11, wherein the selection of the first subclass for the first data asset is performed with a machine learning model, the machine learning model being configured to analyze the format and schema of the first data asset and select a subclass among the hierarchy of classes.
24. The method of embodiment 23, wherein the selection of the first subclass for the first data asset further comprises selecting the first subclass with a trained decision tree machine learning model.
25. The method of embodiment 11, wherein a machine learning model selects the first subclass and selects the second subclass.
26. The method of embodiment 11, further comprising; recording a modification made to the first data asset; and retrieving a version of the first data asset without the modification in response to a user request.
27. The method of embodiment 26, further comprising: applying cryptographic hash pointers to the first data asset, wherein the cryptographic hash pointers references a previous version of the first data asset to form a tamper evident record.
28. The method of embodiment 11, further comprising: receiving, from a user, a confirmation of the first subclass or a selection of another subclass by the user, wherein the subclass confirmation or selection by the user controls which subclass is used for the transformation of data assets.
29. The method of embodiment 11, wherein the computer system is configured to store user account data that specifies roles and permissions of users.
30. The method of embodiment 11, wherein the transforming the first data asset comprises steps for transforming the first data asset.
31. A tangible, non-transitory, machine-readable medium storing instructions that when executed by one or more computers effectuate the operations of any one of embodiments 1-30.
32. A system, comprising: one or more processors; and memory storing instructions that when executed by the one or more processors cause the one or more processors to execute any one of embodiments 1-30.

What is claimed is:

1. A method, comprising:
obtaining, with a computer system, clinical trial data in diverse formats and schemas;
transforming, with the computer system, the clinical trial data into a unified format and a unified schema to produce transformed clinical trial data, wherein transforming comprises:
obtaining a hierarchy of dataset classes, wherein:
classes further from a root dataset class in the hierarchy inherit from dataset classes closer to the root dataset class in the hierarchy, and the dataset classes map a first set of variables in input data to concepts used in data analysis, a second set of variables in input data to concepts used in visualization, and a third set of variables in input data to concepts used in data operations;
the root dataset class maps a patient identifier in input data to a subject identifier in the unified schema, wherein the root dataset class is configured to map different identifiers of the same patient in different input data to the same subject identifier in the unified schema;
an event dataset class inherits from the root dataset class and maps input data with diverse formats and schemas characterizing events to event fields in the unified schema, in the unified format;
an adverse event dataset class inherits from the event dataset class and maps input data with diverse formats and schemas characterizing adverse to adverse event fields in the unified schema, in the unified format; and
a subject level dataset class inherits from the root dataset class and is configured to produce records indexed by the subject identifier in the unified schema, the subject level dataset class being configured to map diverse formats and schemas input data characterizing treatment variables, treatment start dates, and treatment end data into corresponding fields in the unified schema, in the unified format;
parsing the clinical trial data into records;
determining which dataset classes correspond to each of the records with the hierarchy of dataset classes; and
transforming each of the records into the unified format and the unified schema with the corresponding dataset classes in the hierarchy of dataset classes; and
storing, with the computer system, the transformed clinical trial data in memory.

2. The method of claim 1, wherein at least some of the dataset classes specify visualization, the method further comprising:
selecting a plurality of visualization choices as a subset of a set of candidate visualization choices based on visualizations specified by dataset classes that correspond to the records.

3. The method of claim 2, wherein the subset comprises visualizations specified by dataset classes from which the dataset classes that correspond to the records inherit in the hierarchy of dataset classes.

4. The method of claim 1, comprising precomputing, before the transforming, inheritance properties of the dataset classes to flatten hierarchical relationships, and using the flattened hierarchical relationships to perform the transforming.

5. The method of claim 1, comprising: precomputing, before the transforming, mappings from the hierarchy of classes and caching the precomputed mappings with a hash map, wherein multiple accesses of the same mapping during the transforming are expedited by the hash map relative to traversing the hierarchy directly.

6. The method of claim 1, wherein the hash map is a nested hash map.

7. The method of claim 1, comprising retrieving, during the transforming, dataset classes with a balanced tree index formed before the transforming.

8. The method of claim 7, wherein the balanced tree index is an AVL (Adelson-Velsky and Landis) tree, a red-black tree, a B-tree, or a B+ tree.

9. the method of claim 1, comprising concurrently determining whether a plurality of dataset classes correspond to a given record among the records formed by parsing the clinical trial data.

10. The method of claim 1, comprising concurrently determining whether a plurality of records correspond to a given record among the records formed by parsing the clinical trial data.

11. A method for transforming clinical data into a data model, comprising:
obtaining, with a computer system, data assets characterizing a plurality of clinical studies, wherein a first data asset among the data assets is in a first format having a first data schema and a second data asset among the data assets is in a second format having a second data schema;
selecting, with the computer system, a first subclass from a hierarchy of classes for the first data asset, wherein the first subclass specifies how to transform the first format and first data schema into a shared data format and a shared data schema;
selecting, with the computer system, a second subclass from the hierarchy of classes for the second data asset, wherein the second subclass specifies how to transform the second format and second data schema into the shared data format and the shared data schema, wherein at least one of the shared data format and the shared data schema or both collectively comprise:
a base dataset class comprising a mapping relating a subject identifier;
an event dataset class inheriting from the base dataset class; and
a subject-level dataset class inheriting from the base dataset class, wherein at least some subclasses in the hierarchy of classes inherit from a shared parent class in the hierarchy of classes, and at least some of the subclasses in the hierarchy of classes inherit from a root class in the hierarchy of classes;
transforming, with the computer system, the first data asset into the shared data format and the shared data schema using the selected first subclass to produce a version of the first data asset in the shared data format and the shared data schema;
transforming, with the computer system, the second data asset into the shared data format and the shared data schema using the selected second subclass to produce a version of the second data asset in the shared data format and the shared data schema;
storing, with the computer system, the version of the first data asset in the shared data format and the shared data schema in memory; and
storing, with the computer system, the version of the second data asset in the shared data format and the shared data schema in memory.

12. The method of claim 11, further comprising:
populating, with the computer system, one or more visualizations based on the transformed first data asset, the visualizations corresponding to at least one of the dataset classes; and
receiving, with the computer system, input from a user through an interactive interface to manipulate the one or more visualizations, wherein the input modifies at least one aspect of the visualizations to reflect updated parameters, filters, or analytical views.

13. The method of claim 12, wherein the one or more visualizations generated by the computer system comprise at least one of: an incidence plot, a swimmer plot, a univariate numeric summary, or a univariate categorical summary.

14. The method of claim 12, wherein the one or more visualizations generated by the computer system comprises: an incidence plot, a swimmer plot, a univariate numeric summary, and a univariate categorical summary.

15. The method of claim 11, wherein the event dataset class of the shared data format and data schema further comprises a mapping of a start variable, an end variable, a name variable, and a classification variable.

16. The method of claim 11, wherein the shared data format and data schema further comprises:
an adverse events dataset class inheriting from the events dataset class, the adverse events dataset class comprising a mapping related to a severity level of an adverse event.

17. The method of claim 11, wherein selecting the first subclass further comprises:
preprocessing, with the computer system, the first data asset to identify indicators of a dataset class and domain; and
determining, with the computer system, that a dataset domain and class is to be used based on the indicators identified.

18. The method of claim 17, wherein the first data asset is transformed by mapping, with the computer system, the first data asset to a set of default mappings of the determined dataset domain and class.

19. The method of claim 17, further comprising:
identifying, within the first transformed data asset, variables corresponding to subject identifiers, event classifications, and subject-level attributes;
creating a subject identifier mapping that links each data record to a subject identifier; and
generating event-specific mappings defining variables related to start and end times, classifications, and descriptions of clinical events.

20. The method of claim 19, further comprising:
accessing, with the computer system, stored transformation data;
comparing, with the computer system, the first data format and first data schema of the first data asset with the data formats and data schemas stored in transformation data; and
determining, with the computer system, that the first data asset is to be transformed according to transformation instructions stored in the transformation data based on the comparison.

21. The method of claim 11, wherein the first data asset has a different schema and a different format from the second data asset.

22. The method of claim 11, wherein the computer system performing the functions of the method is implemented in a cloud-based system architecture, the cloud-based system architecture comprising a plurality of client-facing interface servers and a plurality of database servers.

23. The method of claim 11, wherein the selection of the first subclass for the first data asset is performed with a machine learning model, the machine learning model being configured to analyze the format and schema of the first data asset and select a subclass among the hierarchy of classes.

24. The method of claim 23, wherein the selection of the first subclass for the first data asset further comprises selecting the first subclass with a trained decision tree machine learning model.

25. The method of claim 11, wherein a machine learning model selects the first subclass and selects the second subclass.

26. The method of claim 11, further comprising;
recording a modification made to the first data asset; and
retrieving a version of the first data asset without the modification in response to a user request.

27. The method of claim 26, further comprising:
applying cryptographic hash pointers to the first data asset, wherein the cryptographic hash pointers references a previous version of the first data asset to form a tamper evident record.

28. The method of claim 11, further comprising:
receiving, from a user, a confirmation of the first subclass or a selection of another subclass by the user, wherein the subclass confirmation or selection by the user controls which subclass is used for the transformation of data assets.

29. The method of claim 11, wherein the computer system is configured to store user account data that specifies roles and permissions of users.

30. The method of claim 11, wherein the transforming the first data asset comprises steps for transforming the first data asset.
